(19) United States
(12) Patent Application Publication
(10) Pub. No.: US 2025/0352522 A1
(43) Pub. Date: Nov. 20, 2025

(54) RAPID INDUCTION WITH MONTHLY INJECTABLE BUPRENORPHINE EXTENDED-RELEASE

(71) Applicant: Indivior UK Limited, Hull (GB)

(72) Inventors: Christian A. HEIDBREDER, North Chesterfield, VA (US); Frank GRAY, London (GB); Celine M. LAFFONT, North Chesterfield, VA (US); Robert L. DOBBINS, North Chesterfield, VA (US)

(21) Appl. No.: 19/170,285

(22) Filed: Apr. 4, 2025

Related U.S. Application Data
(63) Continuation of application No. 18/920,176, filed on Oct. 18, 2024.
(60) Provisional application No. 63/544,747, filed on Oct. 18, 2023.

(51) Int. Cl. A61K 31/4355; A61P 25/36

(52) U.S. Cl. CPC A61K 31/4355; A61P 25/36

(57) ABSTRACT

The disclosure provides methods of treating opioid use disorder in a human by rapid induction with monthly injectable extended release buprenorphine, and to rapid induction dosage regimens for treating opioid use disorder. The methods provide increased treatment retention compared to standard of care methods.

### CROSS-REFERENCE TO RELATED APPLICATIONS

The present application claims priority to U.S. application Ser. No. 18/920,176, filed Oct. 18, 2024, and U.S. Provisional Patent Application No. 63/544,747, filed Oct. 18, 2023, the disclosures of which are incorporated herein.

### TECHNICAL FIELD

The disclosure relates to methods of treating opioid use disorder in a human by rapid induction with monthly injectable buprenorphine extended-release, and to rapid induction dosage regimens for treating opioid use disorder.

### BACKGROUND

Opioids are powerful medications and drugs which are prescribed following injury or surgery to relieve moderate-to-severe pain to enable activity, or for disease conditions including cancer. Opioids include substances such as morphine, fentanyl, codeine, hydrocodone, oxycodone, oxymorphone, hydromorphone, tapentadol, and methadone. Due to the widespread availability and variety of prescription opioid products, the number of opioid prescriptions is significantly high (153 million in 2019) in the United States alone.

The inappropriate use of opioids often causes serious problems such as addiction, abuse, overdose, and dependence on opioids, known as opioid use disorder (“OUD”). OUD is a chronic, relapsing disease associated with an elevated risk of mortality and morbidity that has been described as one of the most challenging forms of addictions. According to the Diagnostic and Statistical Manual for Mental Disorders, 5th Edition (“DSM-5”), OUD is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. OUDs affect over 16 million people worldwide, over 2.1 million in the United States among them, and cause over 120,000 deaths worldwide annually attributed to opioids. The U.S. Department of Health and Human Services reports that overdose deaths involving opioids increased 519.38% from 1999 to 2019. In addition to these serious issues, the use of opioids can cause a number of side effects including increased opioid tolerance, symptoms of withdrawal when discontinued, increased sensitivity to pain, nausea, and/or depression.

Opioid receptors are located in both the central nervous system (“CNS”) and the periphery. In the CNS, they are found in high concentrations in the limbic system and the spinal cord. The natural ligands for the opioid receptors are a group of neuropeptides known as endorphins. Opioid analgesics mimic the action of these natural ligands but have a more prolonged action as they are not subject to rapid local metabolism. Three major opioid receptor subclasses have been identified: μ-, κ-, and δ-.

Buprenorphine (“BUP”) is a semisynthetic derivative of thebaine and is a mixed partial agonist opioid receptor modulator with the chemical formula C₂₉H₄₁NO₄, and chemical name 21-cyclopropyl-7α-[(S)-1-hydroxy-1,2,2-trimethylpropyl]-6,14-endo-ethano-6,7,8, 14-tetrahydrooropavine. Buprenorphine is a partial opioid agonist at the μ-opioid receptor with antagonist properties at the K-receptor, and has been widely used for Medication-Assisted Treatment of OUD. In contrast to a full agonist, buprenorphine at the μ-receptor has less maximal euphoric effect, and a ceiling on its respiratory depressant effects. By binding to μ-opioid receptors in the brain, buprenorphine reduces craving for opioids and opiate withdrawal symptoms, minimizing the need of opioid-dependent patients to use illicit opiate drugs. Buprenorphine is the active ingredient in immediate-release formulations such as transmucosal (“TM”) formulations like SUBOXONE®, and long-acting injectable formulations such as SUBLOCADE®. SUBLOCADE® is the first monthly, extended-release buprenorphine injection (“BUP-XR” or “RBP-6000”) approved in the U.S. for treatment of moderate-to-severe OUD. SUBLOCADE® was designed for dosing regimens selected to ensure patients are exposed to safe and therapeutic levels throughout the month, with no drop in concentrations that would trigger re-emergence of opioid withdrawal, craving, or potential relapse to opioid use.

Currently, standard of care induction or standard induction (“SoC” or “SI”) methods of treating OUD include a stepwise dosing approach for administration of TM buprenorphine. On day 1, TM buprenorphine is typically given at an initial dose of 2 to 4 mg, with upwards titration in 2 to 4 mg increments up to a maximal daily dose of 8 to 16 mg. On day 2, TM buprenorphine dose is adjusted based on patient's evaluation. Typically, a single daily dose of 12 or 16 mg is administered. Administration of BUP-XR begins after a minimum of seven (7) days of induction and dose stabilization with TM buprenorphine. However, there is a need for improved methods for treating OUD with buprenorphine, especially in high-risk populations. For example, patients who use intravenous drugs, very high doses of opioids, or highly potent synthetic opioids have an increased risk of treatment failure and opioid overdose with SI methods. Therefore, dosage regimens and treatment methods that reduce the risk of treatment failure compared to current dosage regimens and treatment methods would be beneficial.

To this end, the present disclosure provides rapid induction (“RI”) dosing regimens for buprenorphine, as well as dosing regimens and treatment methods that offer, among other benefits, optimal ways to reach and maintain buprenorphine dosages for the treatment of opioid dependence in patients, particularly in high-risk opioid users, compared to SI dosing regimens.

### SUMMARY

The disclosure relates to methods of treating OUD in a human by RI with injectable BUP-XR, for example SUBLOCADE®, and to RI dosing regimens for treating opioid use disorder in a human.

In some embodiments, the disclosure relates to methods of treating OUD in a human in need thereof, the methods comprising administering TM buprenorphine hydrochloride to the human and, within no more than about 24 hours, such as no more than about 12 hours, administering BUP-XR to the human.

In some embodiments, the disclosure relates to methods of treating OUD in a human in need thereof, the methods comprising:
a) administering a composition comprising about 4 mg of TM buprenorphine hydrochloride (e.g. SUBOXONE® or any other commercially available equivalent drug product containing buprenorphine) to the human on day 1, and
b) administering about 300 mg of BUP-XR (e.g. SUBLOCADE® or any other commercially available equivalent monthly drug product such as BRIXADI®) by subcutaneous injection at least about one hour later.

In other embodiments, the disclosure provides methods of treating OUD in a human in need thereof, the methods comprising:
a) administering a composition comprising about 4 mg of TM buprenorphine hydrochloride to the human on day 1,
b) administering about 300 mg of BUP-XR by subcutaneous injection one at least about one hour later,
c) administering a second dose of about 300 mg of BUP-XR to said human by subcutaneous injection 2 as early as about day 8 or up to about one month after injection 1, and
d) administering from about 100 mg to about 300 mg of BUP-XR monthly to the human, e.g. about 100 mg or about 300 mg, by subcutaneous injection 3 starting about one about month from administration of injection 2 and continuing approximately monthly as long as needed.

In various embodiments, the RI methods described herein provide at least one of the following results:
(i) completion of steps (a) through (b) has a retention rate superior to SI at an endpoint of injection 2;
(ii) completion of steps (a) though (b) does not increase Precipitated Opioid Withdrawal (“POW”) relative to SI;
(iii) for patients experiencing POW:
   (iii)(a) retention at injection 1 is higher after completion of step (a) with RI than for SI;
   (iii)(b) retention at injection 2 is higher after completion of steps (a) through (b) than for SI; and/or
   (iii)(c) retention at injection 3 is higher after completion of steps (a) through (c) than for SI;
(iv) the Clinical Opiate Withdrawal Score (“COWS”) from completion of steps (a) through (b) during RI is about the same as or lower than for SI;
(v) treatment-emergent adverse events (“TEAEs”) up to the second injection in steps (a) through (b) are comparable to the TEAEs up to the second injection for SI; and/or
(vi) treatment-emergent adverse events (TEAEs) up to the third injection in steps (a) through (c) are comparable to the TEAEs up to the third injection for SI.

Thus, the disclosure provides RI dosing regimens, as well as treatment methods that reduce the risk of treatment failure compared to current dosing regimens and treatment methods using buprenorphine.

The present disclosure may be understood more readily by reference to the following detailed description and the illustrative Example included herein. Unless defined otherwise, all technical and scientific terms used herein have the meaning commonly understood by one of ordinary skill in the relevant art.

### BRIEF DESCRIPTION OF THE DRAWINGS

FIGS. 1A-1B are mean (+SD) buprenorphine plasma concentration-time profiles in 300 mg/100 mg (1A) and 300 mg/300 mg (1B) dosing regimens. Both dosing regimens start with two monthly injections of 300 mg followed by 4 monthly injections of either 100 mg (1A) or 300 mg (1B).

FIG. 2 shows a study design for comparing the efficacy, safety, and tolerability of 100 mg and 300 mg maintenance doses of BUP-XR in treatment-seeking participants with moderate to severe OUD and high-risk opioid use. This study included an open-label sub-study comparing rapid induction vs. standard induction onto BUP-XR.

FIG. 3 shows COWS scores on induction day of the sub-study (interim data analysis).

FIG. 4 is a graph showing time to treatment discontinuation in the sub-study (final data analysis).

FIG. 5 shows COWS scores on induction day of the sub-study (final data analysis).

FIG. 6 is a flow chart for the rapid induction arm.

FIG. 7 shows a pharmacokinetic simulation for a 300/100-mg BUP-XR dosing regimen with administration of the second dose at Week 2 compared with traditional dosing at Week 5.

### DESCRIPTION

The disclosure relates to methods of treating OUD in a human patient by RI with administration of TM buprenorphine followed by injectable BUP-XR, followed, in some embodiments, by monthly maintenance doses of injectable BUP-XR. The methods may improve treatment retention compared to methods employing SI induction.

In various aspects, the patient is one who uses opioids via an injection route. In various aspects, the patient is from high-risk populations. In various aspects, the patient is one who uses intravenous drugs, very high doses of opioids, or highly potent synthetic opioids. Those using very high doses or highly potent synthetic opioids can have an increased risk of treatment failure and opioid overdose. In aspects, the patient is black or African American who self-identifies their race as black or African American. In aspects, the patient is white who self-identifies their race as white. In aspects, the patient is Hispanic who self-identifies their race as Hispanic. In aspects, the patient is Asian who self-identifies their race as Asian. In aspects, the patient is a combination of two or more races who self-identify their race as the combination of two or more races.

In various aspects, the methods comprise administering about 2 mg to about 4 mg TM buprenorphine on day 1 of treatment, followed by subcutaneous administration of about 100 mg to about 300 mg of BUP-XR (injection 1) within no more than about 24 hours, for example within about 1 hour to about 12 hours, within about 1 hour to about 6 hours, or within about 1 hour to about 3 hours. The methods may further comprise administration of a second subcutaneous injection of about 100 mg to about 300 mg of BUP-XR (injection 2) on day 8. In various aspects, the methods further comprise monthly subcutaneous administration of about 100 mg to about 300 mg of BUP-XR (injections 3+) for a prescribed time period.

For example, the methods may comprise administering about 4 mg TM buprenorphine on day 1 of treatment, followed by subcutaneous administration of about 300 mg of BUP-XR (injection 1) within no more than about 24 hours, for example within about 1 hour to about 12 hours, within about 1 hour to about 6 hours, or within about 1 hour to about 3 hours. The methods may optionally further comprise administration of a second subcutaneous injection of about 100 mg to about 300 mg of BUP-XR (injection 2) on day 8 of treatment.

As a further example, the methods may comprise administering about 4 mg TM buprenorphine on day 1 of treatment, followed by subcutaneous administration of about 100 mg to about 300 mg of BUP-XR (injection 1) within no more than about 24 hours, for example within about 1 hour to about 12 hours, within about 1 hour to about 6 hours, or within about 1 hour to about 3 hours; administering a second subcutaneous injection of about 100 mg to about 300 mg of BUP-XR (injection 2) on day 8; and administering one or more monthly subcutaneous injections of about 100 mg to about 300 mg of BUP-XR (injections 3+) for a prescribed time period.

As yet a further example, the methods may comprise administering about 4 mg TM buprenorphine on day 1 of treatment, followed by subcutaneous administration of about 300 mg of BUP-XR (injection 1) within no more than about 24 hours, for example within about 1 hour to about 12 hours, within about 1 hour to about 6 hours, or within about 1 hour to about 3 hours; administering a second subcutaneous injection of about 100 mg to about 300 mg of BUP-XR (injection 2) on day 8; and administering one or more monthly subcutaneous injections of about 100 mg or about 300 mg of BUP-XR (injections 3+) for a prescribed time period.

In various embodiments, the total number of subcutaneous injections of BUP-XR administered to the patient in the methods according to the disclosure may be 1, or may be more than 1. By way of example only, the total number of subcutaneous injections of BUP-XR in the methods according to the disclosure may range from 1 to 10, from 1 to 9, from 1 to 8, from 1 to 7, from 1 to 6, from 1 to 5, from 1 to 4, from 1 to 3, from 1 to 2, from 2 to 10, from 2 to 9, from 2 to 8, from 2 to 7, from 2 to 6, from 2 to 5, from 2 to 4, from 2 to 3, from 3 to 10, from 3 to 9, from 3 to 8, from 3 to 7, from 3 to 6, from 3 to 5, or from 3 to 4.

As used herein, the term “BUP” refers to buprenorphine.

The term “extended-release buprenorphine” or “BUP-XR” refers to a buprenorphine formulation that is formulated to last longer in the body than immediate-release buprenorphine, so that the intended benefit lasts over a period of time of about one month. Thus, BUP-XR is understood as being formulated for monthly administration. As non-limiting examples, SUBLOCADE® and RBP-6000, in which the active ingredient is buprenorphine free base, may be used. In some aspects, BUP-XR contains about 300 mg buprenorphine free base. In other aspects, BUP-XR contains about 100 mg buprenorphine free base. In some aspects, a composition comprising about 100 mg BUP-XR and a composition comprising about 300 mg BUP-XR each comprises (i) about 18 wt % of buprenorphine free base; (ii) about 32 wt % of a poly(DL-lactide-co-glycolide) copolymer; and (iii) about 50 wt % of N-methyl-2-pyrrolidone. In other aspects, BUP-XR can be any commercially available product having buprenorphine free base or a salt thereof as the active ingredient.

In various aspects, the BUP-XR formulations comprise about 300 mg of buprenorphine, which is defined as 295 mg to 305 mg.

In various aspects, BUP-XR formulations comprise about 100 mg of buprenorphine, defined as 95 mg to 105 mg.

In various aspects, about 18 wt % of buprenorphine free base is defined as from 17 wt % to 19 wt %; about 32 wt % of a poly(DL-lactide-co-glycolide) copolymer is defined as from 31 wt % to 33 wt %; and about 50 wt % of N-methyl-2-pyrrolidone is defined as from 48 wt % to 52 wt %.

A dosing of BUP-XR that is “monthly” means that the BUP-XR is administered one time in one month, and at least a second time one month later. “One month” is defined as about 28 days to about 31 days. For example, the BUP-XR can be administered the first time on January 1, the second time on February 1, the third time on March 1, etc. The terms “monthly” and “four weeks” may be used interchangeably herein.

As used herein, “TM BUP” and “TM buprenorphine” refer to transmucosal buprenorphine formulations, such as Indivior's product SUBOXONE® that contains buprenorphine hydrochloride.

With reference to the Diagnostic and Statistical Manual for Mental Disorders, 5th Edition, American Psychiatric Association, 2013 (also referred to herein as DSM5), “opioid use disorder” is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. In aspects, the opioid use disorder is moderate opioid use disorder. “Moderate opioid use disorder” is defined by reference to the DSM5 Opioid Use Disorder Checklist (ICD-9-CM code 304.00 or ICD-10-CM code F11.20) as having the presence of 4 or 5 symptoms indicated in the DSM5 Opioid Use Disorder Checklist. In aspects, the opioid use disorder is severe opioid use disorder. “Severe opioid use disorder” is defined by reference to the DSM5 Opioid Use Disorder Checklist (ICD-9-CM code 304.00 or ICD-10-CM code F11.20) as having the presence of 6 or more symptoms indicated in the DSM5 Opioid Use Disorder Checklist. In aspects, the opioid use disorder is moderate-to-severe opioid use disorder. Moderate-to-severe opioid use disorder refers to the presence of 4 or more symptoms indicated in the DSM5 Opioid Use Disorder Checklist. In aspects, the opioid use disorder is mild opioid use disorder. “Mild opioid use disorder” is defined by reference to the DSM5 Opioid Use Disorder Checklist (ICD-9-CM code 305.50 or ICD-10-CM code F11.10) as having the presence of 2 or 3 symptoms indicated in the DSM5 Opioid Use Disorder Checklist. In aspects, the opioid use disorder is mild-to-moderate opioid use disorder. Mild-to-moderate opioid use disorder refers to the presence of 2 to 5 symptoms indicated in the DSM5 Opioid Use Disorder Checklist (https://www.asam.org/docs/default-source/education-docs/dsm-5-dx-oud-8-28-2017.pdf). In aspects, “treating opioid use disorder” encompasses one or more of (i) reducing opioid withdrawal symptoms, (ii) eliminating opioid withdrawal symptoms, (iii) reducing opioid craving, (iv) eliminating opioid craving, (v) reducing illicit opioid use, (vi) eliminating illicit opioid use, and (vii) inducing opioid abstinence.

The term “opioid withdrawal signs and symptoms” includes one or more signs and symptoms associated with withdrawal from opioids. Such signs and symptoms can include one or more of the following: agitation, anxiety, muscle aches, increased tearing, insomnia, runny nose, sweating, yawning, abdominal cramping, diarrhea, dilated pupils, goose bumps, nausea, and vomiting. Opioid withdrawal symptoms can begin to occur from a few hours to a few days after the last intake of an opioid, with the time being dependent on the opioid, the person's metabolism, and other factors.

The term “administering” includes administration of the formulations described herein to the patient, and can include implantation of a slow-release device in the patient. In aspects, “administering” BUP-XR refers to parenteral administration, for example subcutaneously injecting the formulations. Parenteral administration includes, for example, intravenous, intramuscular, intra-arterial, intradermal, intrathecal, subcutaneous, intraperitoneal, intraventricular, or intracranial. In aspects, “administering” TM BUP refers to buccal or sublingual administration.

As used herein, the terms “treat,” “treating,” or “treatment” include at least one of (i) preventing a condition from occurring (prophylaxis); (ii) inhibiting a condition and/or arresting its development; and (iii) relieving symptoms associated with a condition.

As used herein, the terms “patient” and “subject” refer to a human.

“Therapeutic levels” of buprenorphine refers to buprenorphine plasma concentrations that can be effective to achieve at least one of the following results: (a) treatment of OUD; (b) suppressing opioid withdrawal symptoms; (c) eliminating opioid withdrawal symptoms; (d) reducing opioid craving; (e) eliminating opioid craving; (f) reducing illicit opioid use; (g) preventing illicit opioid use; (h) inducing opioid abstinence; or (i) a combination of two or more of the foregoing. “Therapeutic levels” can also be described in terms of steady-state minimum buprenorphine plasma concentration levels, steady-state average buprenorphine plasma concentration levels, and steady-state maximum buprenorphine plasma concentration levels, all of which are described in more detail herein.

### EXAMPLE

Implementation of the present disclosure is provided by way of the following Example. The Example serves to illustrate the technology without being limiting in nature.

This Example describes a study comparing rapid induction (RI) and standard induction (SI) for initiation of monthly injectable BUP-XR in treatment-seeking adult participants with OUD and high-risk opioid use (NCT04995029). The study compares the efficacy, safety, and tolerability of two maintenance dose levels of BUP-XR, 300 mg and 100 mg administered every four weeks, in treatment-seeking participants with moderate to severe OUD and high-risk opioid use (those who use opioids via an injection route and/or use high doses of opioids and/or use highly potent synthetic opioids) that may benefit from the higher 300 mg maintenance dose.

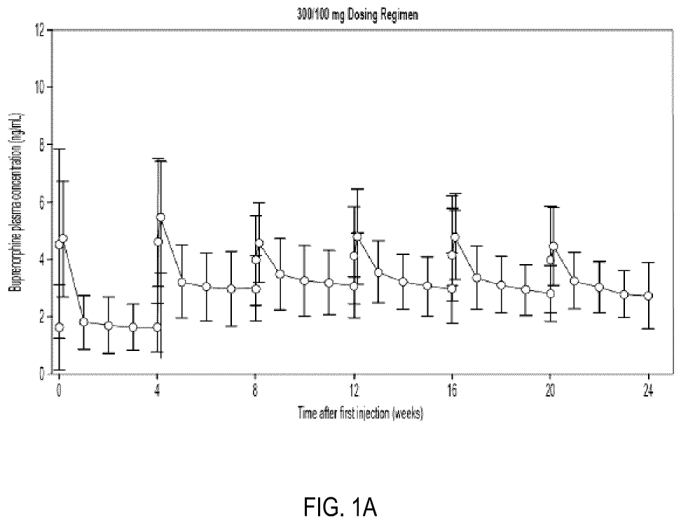

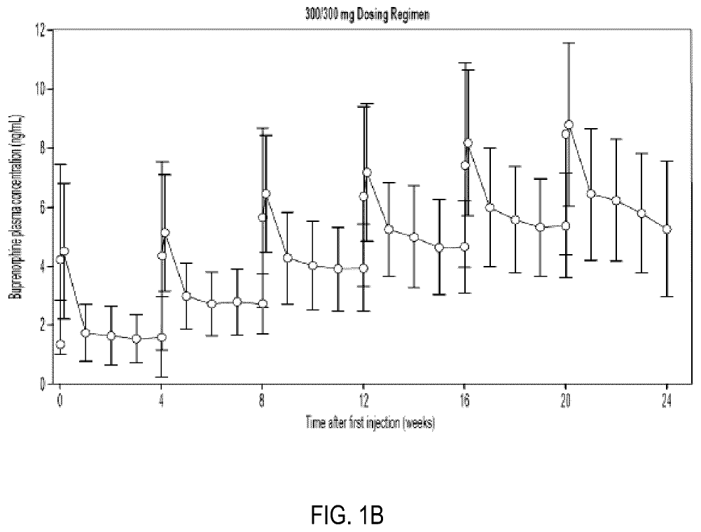

In the study, two dosing regimens of buprenorphine extended-release (BUP-XR) were evaluated. The first regimen shown in FIG. 1A (n=203) consisted of two monthly injections of 300 mg BUP-XR followed by four monthly injections of 100 mg BUP-XR. The second regimen shown in FIG. 1B (n=201) involved six monthly injections of 300 mg BUP-XR. The data in FIGS. 1A-1B were analyzed and presented with standard deviation (SD) to account for variability in the results.

The study also included an open-label induction sub-study (“the sub-study” or “OLIS”), the primary objective of which was to compare treatment retention of participants initiated onto BUP-XR using RI compared to SI.

### Study Plan

### Study Design

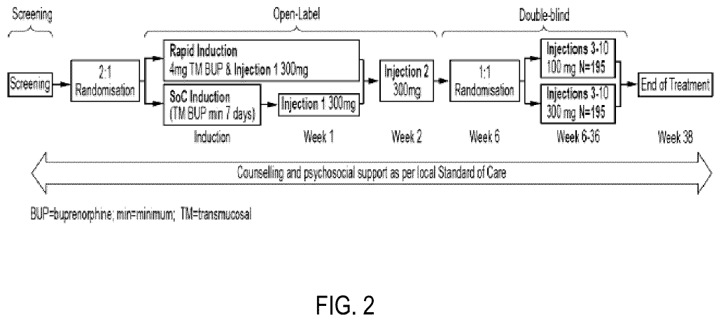

The overall study design is shown in FIG. 2. In FIG. 2, week 1 is time zero (i.e. there is no week 0), so week 6 is 5 weeks after day 1. Participants were randomized at a 2:1 ratio to the RI or SI arms. SUBLOCADE® was used as BUP-XR for this study. TM buprenorphine was selected by the investigator per local prescribing guidelines, and administered either under the tongue (sublingual) or between the gum and cheek (buccal).

Treatment Period: The treatment period began when the participant received TM buprenorphine and ended when the week 38 end of treatment (“EOT”) visit or early termination (“ET”) visit was completed.

Open-label Treatment Period: The Open-label Treatment Period (“OLTP”) began when the first TM buprenorphine dose is given and ends immediately prior to a participant receiving double-blind BUP-XR injection 3.

Randomization and Double-Blind Treatment Period: The randomized Double-blind Treatment Period (“DBTP”) began when the participant received randomized treatment at week 6 (injection 3) and ended when the week 38 EOT visit or ET visit was completed.

### Eligibility

Eligible participants were selected based on the inclusion criteria described hereafter during the screening. All eligible participants signed the informed consent form (ICF) and were able and willing to comply with the requirements and restrictions described hereafter. Proper counselling and psychosocial support as per the local standards were provided to the patients. Eligible participants were selected based on the following inclusion criteria during screening:
1. Had signed the ICF and was willing and able to comply with the requirements and restrictions therein;
2. Was 18 years of age or older at the time of signing the ICF;
3. Currently met DSM-5 criteria for moderate or severe OUD;
4. Had a history of OUD as defined by documented medical history of OUD for at least 90 days immediately prior to providing informed consent;
5. Met at least one of the following criteria for high-risk opioid use at the screening visit:
   (a) using opioids via the injection route for an average of 5 or more days per week in the last four weeks; or
   (b) using at least 500 mg IV heroin equivalent (e.g., 1250 mg intravenous (IV) morphine) or self-reported use of any dose of highly potent synthetic opioids (fentanyl and analogues excluding transdermal patches) for an average of
5 or more days per week in the last four weeks by any route;
6. Was seeking medication for the treatment of OUD;
7. Was an appropriate candidate for opioid partial-agonist medications for OUD in the opinion of the Investigator or medically qualified sub-Investigator;
8. Agreed not to use buprenorphine-containing products other than those administered as part of study treatment for the duration of the treatment period; and
9. If female, was not pregnant, not lactating and, if of childbearing potential, agreed not to become pregnant while in the study and to use medically acceptable means of contraception while in the study.

Participants were not eligible for inclusion in the study if any of the following exclusion criteria applied:
1. Had a current diagnosis, other than OUD, requiring chronic opioid treatment;
2. Has a concurrent primary substance use disorder, as defined by DSM-5 criteria, other than opioid, tobacco, cannabis, or alcohol use disorders;
3. Met DSM-5 criteria for severe alcohol use disorder;
4. Had significant traumatic injury or major surgical procedure (as defined by the Investigator) within 30 days prior to the first dose of BUP-XR or still recovering from prior injury or surgery;
5. Had congenital long QT syndrome, history of prolonged QT in the 3 months prior to screening, or history of medications or other factors that are at risk for Torsades de Pointes;
6. Had abdominal area unsuitable for sub-cutaneous injections (e.g., nodules, scarring, lesions, excessive pigment, etc.);
7. Had a history of suicidal ideation within 30 days prior to informed consent or history of a suicide attempt in the 6 months prior to informed consent;
8. Had uncontrolled intercurrent illness including, but not limited to, a medical or psychiatric illness/social situation that would limit compliance with study requirements or compromise the ability of the participant to provide written informed consent;
9. Had any other active medical condition, organ disease, or concurrent medication or treatment that may either compromise participant safety or interfere with study endpoints;
10. Had total bilirubin of ≥1.5×upper limit of normal (“ULN”) (with direct bilirubin>1.3 mg/dL), ALT≥3×ULN, AST≥3×ULN, serum creatinine>2×ULN, or international normalized ratio (“INR”)>1.5×ULN at screening;
11. Had known allergy or hypersensitivity to buprenorphine or any component of the ATRIGEL delivery system;
12. Was undergoing concurrent treatment, or has had prior treatment, with any long-acting form of a buprenorphine-containing product in the past 2 years, or if >2 years had a positive urine drug screen (“UDS”) for buprenorphine at screening; treatment with oral buprenorphine, oral naltrexone, or methadone products within 14 days prior to consent; or treatment with depot naltrexone within 3 months prior to consent;
13. Was undergoing concurrent treatment with another investigational agent or enrollment in another clinical study (except for an observational study) or treatment with another investigational agent within 30 days prior to screening;
14. Was undergoing concurrent treatment with medications contraindicated for use with buprenorphine as per local prescribing information;
15. Was under court order requiring treatment for OUD;
16. Was a member of site staff and/or has a financial interest in the sponsor, or is an immediate family member of either the site staff and/or employee of the sponsor, directly involved in the study; or
17. Had any pending legal status or pending legal action that could prohibit full participation in or compliance with study procedures.

A total of 1434 participants were screened and 785 were randomized. The top reasons for screen failure included 410 participants (28.6%) who were unable to comply with study requirements and did not return after screening, 49 participants (3.4%) with liver function test issues, and 39 participants (2.7%) who did not meet the criteria for high-risk opioid users. Table 1 shows the disposition of all participants screened.

TABLE 1
Disposition of Participants

| | Overall | |
|---|---|---|
| | SoC | RI |
| Screened | 1434 | |
| Randomised | 785 (54.7%) | |
| | 267 | 518 |

Of the 785 randomized participants, 729 (92.9%) were included in the Safety Full Analysis Set (“SFAS”). Of the 729 participants in the SFAS, 6 non-eligible participants were discontinued after randomization. Therefore, 723 participants were evaluable for treatment retention, which formed the basis for the primary efficacy analysis. Table 2 shows the demographic and baseline characteristics of SFAS participants.

TABLE 2
Demographics and Baseline Characteristics

| | Fentanyl Positive | | Fentanyl Negative | | Total | | |
|---|---|---|---|---|---|---|---|
| | (N = 563) | | (N = 166) | | (N = 729) | | |
| | SoC | RI | SoC | RI | SoC | RI | TOTAL |
| | N = 196 | N = 367 | N = 59 | N = 107 | N = 255 | N = 474 | N = 729 |
| Age, years, Mean (SD) | 39.5 (9.29) | 40.0 (10.46) | 43.3 (10.81) | 44.2 (10.82) | 40.4 (9.77) | 40.9 (10.68) | 40.7 (10.37) |
| Sex, Male, n (%) | 10 [symbol] (54.1) | 215 (58.6) | 28 (47.5) | 57 (53.3) | 134 (52.5) | 272 ( [symbol] 7.4) | 406 (55.7) |
| Race, n (%) | | | | | | | |
| Black or African American | 23 (11.7) | 5 [symbol] (14.4) | 14 (23.7) | 32 (29.9) | 37 (14.5) | 85 (17.9) | 122 (16.7) |
| White | 165 (84.2) | 289 (78.7) | 42 (71.2) | 69 (64.5) | 207 (81.2) | 358 (75.5) | 565 (77.5) |
| Other | 8 (4.1) | 25 (6.9) | 3 (5.1) | 6 (5.6) | 11 (4.3) | 31 (6.6) | 42 (5.8) |
| Opioid Lifetime Use, years (SD) | 14.3 (9.51) | 14.4 (9.52) | 15.9 (8.86) | 15.9 (10.53) | 14.6 (9.37) | 14.8 (9.77) | 14.7 (9. [symbol] 2) |
| Opioid Use Last 4 Weeks, days (SD) | 27. [symbol] (1.22) | 27.8 (0.95) | 27.6 (1.46) | 27.7 (1.51) | 27.7 (1.28) | 27.8 ( [symbol] .10) | 27. [symbol] (1.17) |
| Main Route Last 4 Weeks, n (%) | | | | | | | |
| Injection | 52 (26.5) | 90 (24.5) | 27 (45.8) | 41 (38.3) | 79 (31.0) | 131 (27. [symbol] ) | 210 (28.8) |
| Smoking | 90 (45.9) | 181 (49.3) | 19 (32.2) | 37 (34.8) | 109 (42.7) | 218 (46.0) | 327 (44.9) |
| Oral | 4 (2.0) | [symbol] (1.4) | 2 (3.4) | 4 (3.7) | 6 (2.4) | 9 (1.9) | 15 (2.1) |
| Snorting | 49 (25.0) | 89 (24. [symbol] ) | 11 (18.6) | 15 (23.4) | 60 (23.5) | 114 (24.1) | 174 (23.9) |
| Other | 1 (0.5) | 2 (0.5) | 0 | 0 | 1 (0.4) | 2 (0.4) | 3 (0.4) |

[symbol]

indicates data missing or illegible when filed

Table 2 shows that the demographic and baseline characteristics were generally similar between treatment arms. Mean ages were 40.4 and 40.9 years in the SI and RI arms, respectively. Most participants in each treatment arm fell into the age range of ≥30 to <45 years (59.6% and 53.2% in the SI and RI arms, respectively) or ≥45 to <60 years (23.5% and 28.1%, respectively). Males comprised 55.7% of the population overall (SoC 52.5% and RI 57.4%). Over three quarters of participants in both arms were white (SoC 81.2% and RI 75.5%). The majority of participants (≥83%) in each treatment arm were not Hispanic or Latino. Screening weight and BMI were also similar across treatment arms. Most participants in each treatment arm were current users of nicotine (SoC 83.1% and RI 86.1%) and xanthine/caffeine (86.7% and 88.4%, respectively); 20.4% and 23.8%, respectively, reported being current users of alcohol.

As also shown in Table 2, demographic and baseline characteristics were generally similar across fentanyl positive or negative subpopulations (based on same-day UDS results), although there was a marginally higher percentage of women of childbearing potential in the fentanyl positive subpopulation (SoC 70.0% and RI 75.0%) compared with the fentanyl negative subpopulation (54.8% and 60.0%, respectively), possibly as a result of that subpopulation being marginally younger (mean ages 39.5 and 40.0 years, respectively) than participants in the fentanyl negative subpopulation (mean ages 43.3 and 44.2 years, respectively).

### Study Procedures

### Induction

As shown in FIG. 2, participants who use opioids via an injection route and/or use fentanyl/high doses of opioids were randomly assigned in a 2:1 ratio to the RI arm or SI arm for the induction phase, which was conducted in an open-label setting. Due to the potential for fentanyl use to impact the response to TM buprenorphine, randomization was stratified according to the same-day UDS result for fentanyl (negative or positive).

In the study, day 1 was the time of injection 1 for both groups (RI and SI); day 8 was the time of injection 2 (8=1+7); and day 36 was the time of injection 3 (36=1+7+28).

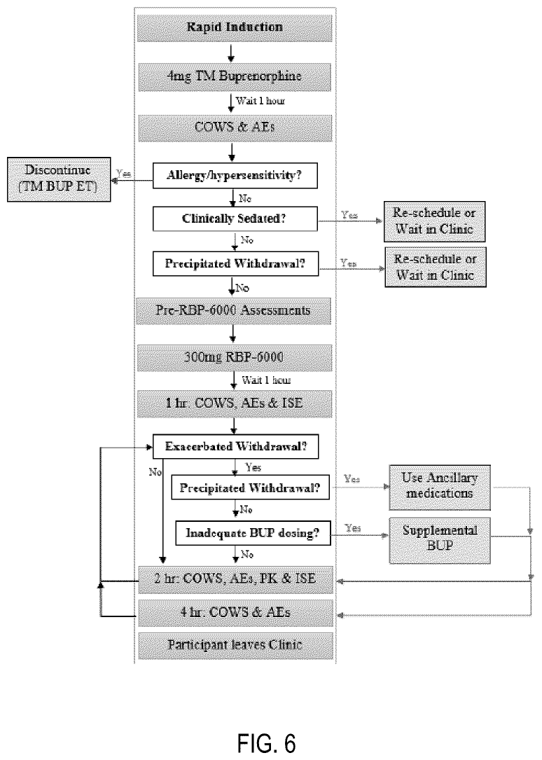

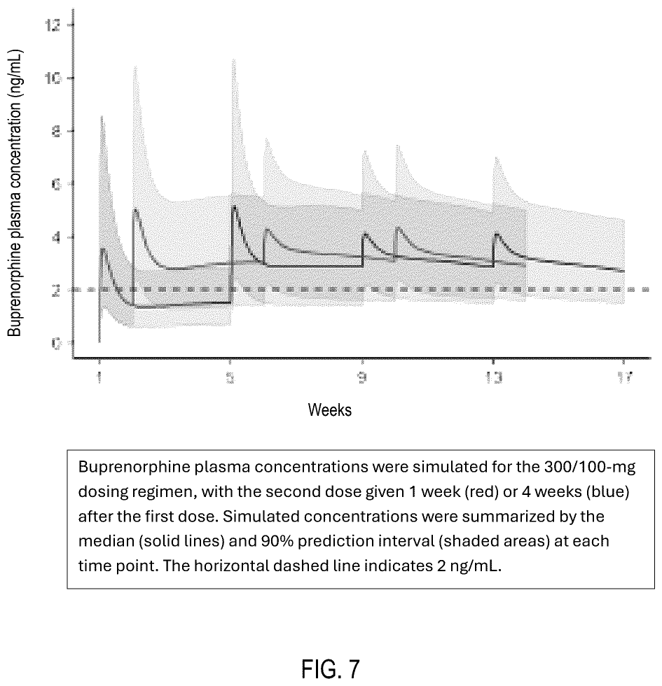

Rapid Induction: The RI arm was designed to initiate BUP-XR treatment following a single dose of TM buprenorphine on the day of induction. On day 1, 4 mg doses of TM buprenorphine were administered to participants. After a minimum of one hour elapsed since the 4 mg dose of TM buprenorphine, the participant was re-assessed for withdrawal symptoms (COWS) and tolerability (AEs). Administration of TM buprenorphine was then followed by subcutaneous injection of 300 mg of BUP-XR (injection 1) to participants meeting eligibility requirements. The flow of activities for participants in the rapid induction arm is shown in FIG. 6.

Standard Induction: In the SI arm, TM buprenorphine was administered for a minimum of 7 days as per applicable product labelling information. In some cases, TM buprenorphine may be administered for 7-14 days. Subsequently, on day 1, 300 mg of BUP-XR was administered to the SI participants meeting eligibility requirements by subcutaneous injection (injection 1). In SI, injection 1 occurred after at least 7 days of induction and dose stabilization with TM buprenorphine.

### Second Dose of BUP-XR

The participants in both the RI and SI arms received another 300 mg BUP-XR (injection 2) on day 8 (+4 days) (i.e. 7+4 days after injection 1).

### Maintenance

Participants were randomized at week 6 prior to injection 3 in a 1:1 ratio to receive maintenance doses of 100 mg or 300 mg BUP-XR. As shown in FIGS. 1A, 1B, and 2, eight (8) additional subcutaneous injections (injections 3-10) of either 100 mg or 300 mg of BUP-XR were then administered on approximately monthly or 4-week bases for both the RI and SI arms.

### Adverse Events

Major safety endpoints include the proportion of participants with at least one TEAE of the following types at any time during the treatment period: any TEAE, drug-related TEAE, treatment-emergent serious AE (SAE), drug-related treatment-emergent SAE, or TEAE leading to treatment discontinuation.

An adverse event (AE) is any untoward medical occurrence in a participant associated with the use of a study drug regardless of the presence of a causal relationship to the study drug. An AE can be any unfavorable and unintended sign (including an abnormal laboratory finding), symptom, or disease (new or exacerbated) temporally associated with a study drug, whether or not considered related to the study drug.

In the study, events meeting the definition of an AE include:
new condition detected after study drug administration even though the AE may have been present prior to receiving study drug,
exacerbation of a pre-existing condition (including intensification of a condition and/or an increase in frequency),
any abnormal laboratory test results or other safety assessments felt to be clinically significant in the opinion of the Investigator (including those that worsen from baseline),
symptoms and/or the clinical sequelae of a suspected interaction,
signs, symptoms or the clinical sequelae resulting from special interest conditions. Overdose per se will not be reported as an AE/SAE unless this is an intentional overdose taken with possible suicidal/self-harming intent. This should be reported regardless of sequelae,
symptoms and/or clinical sequelae resulting from lack of efficacy will be reported if they fulfil the definition of an AE, and
symptoms and/or clinical sequelae that resulted in intervention.

In the study, events that do not meet the definition of an AE include:
the disease/disorder being studied, or expected progression, signs, or symptoms of the disease being studied, unless more severe than expected for the participant's condition,
medical or surgical procedures; the condition that leads to the procedure is an AE,
situations where an untoward medical occurrence did not occur (e.g., social and/or convenience admission to a hospital, hospitalization for elective surgery, hospitalization for observation in the absence of an AE), and
anticipated day-to-day fluctuations of pre-existing disease(s) or condition(s) present or detected at the start of the study that do not worsen.

### Open-Label Induction Sub-Study

A randomized sub-study, nested within the open-label phase of the study, evaluated treatment retention with SI or RI induction to BUP-XR treatment in treatment-seeking participants who frequently injected opioids or used fentanyl/high doses of opioids. The primary objective of the sub-study was to compare treatment retention of participants following RI or SI onto BUP-XR treatment.

Qualified participants who met all of the inclusion criteria, none of the exclusion criteria, and all of the TM buprenorphine dosing criteria were randomized at a 2:1 ratio to either RI or SI. Table 3 below shows demographics and baseline characteristics for the sub-study participants.

TABLE 3
Sub-Study Participants

| | Fentanyl POS Subpopulation | | | Fentanyl NEG Subpopulation | | | Total | | |
|---|---|---|---|---|---|---|---|---|---|
| | (N = 96) | | | (N = 41) | | | (N = 137) | | |
| | SI | RI | Total | SI | RI | Total | SI | RI | Total |
| | (N = 35) | (N = 61) | (N = 96) | (N = 13) | (N = 28) | (N = 41) | (N = 48) | (N = 89) | (N = 137) |
| Age, years, Mean (SD) | 42 (12) | 44 (11) | 44 (11) | 44 (14) | 45 (11) | 45 (12) | 43 (12) | 44 (11) | 44 (11) |
| Sex, Male, n (%) | 13 (37) | 42 (69) | 55 (57) | 9 (69) | 19 (68) | 28 (68) | 22 (46) | 61 (69) | 83 (61) |
| Race, n (%) | | | | | | | | | |
| Black or African American | 10 (29) | 18 (30) | 28 (29) | 4 (31) | 13 (46) | 17 (42) | 14 (29) | 31 (35) | 45 (33) |
| White | 24 (69) | 42 (69) | 66 (69) | 9 (69) | 15 (54) | 24 (59) | 33 (69) | 57 (64) | 90 (66) |
| Other | 1 (3) | 1 (2) | 2 (2) | 0 | 0 | 0 | 1 (2) | 1 (1) | 2 (2) |
| Opioid Lifetime Use, years | 13 (10) | 18 (11) | 16 (11) | 18 (13) | 16 (8) | 17 (10) | 15 (11) | 17 (10) | 17 (10) |
| Opioid Use Last 4 Weeks, days | 27 (2) | 28 (0) | 28 (1) | 28 (0) | 27 (2) | 28 (2) | 28 (2) | 28 (1) | 28 (2) |
| Main Route Last 4 Weeks, n (%) | | | | | | | | | |
| Injection | 11 (31) | 15 (25) | 26 (27) | 9 (69) | 14 (50) | 23 (56) | 20 (42) | 29 (33) | 49 (36) |
| Smoking | 10 (29) | 22 (36) | 32 (33) | 1 (8) | 7 (25) | 8 (20) | 11 (23) | 29 (33) | 40 (29) |
| Oral | 1 (3) | 1 (2) | 2 (2) | 1 (8) | 3 (11) | 4 (10) | 2 (4) | 4 (5) | 6 (4) |
| Snorting | 13 (37) | 23 (28) | 36 (38) | 2 (15) | 4 (14) | 6 (15) | 15 (31) | 27 (30) | 42 (31) |

Among the 137 sub-study participants, fentanyl positive (96) and fentanyl negative (41) subpopulations were categorized. Each fentanyl positive or negative participant was randomly assigned in a 2:1 ratio to RI or SI. As Table 3 shows, 13 male participants (37% of total) of the fentanyl positive subpopulation were assigned to SI, and 42 male participants (69% of total) of the fentanyl positive subpopulation were assigned to RI. Further, 9 male participants (69% of total) of the fentanyl negative subpopulation were assigned to SI, and 19 male participants (68% of total) of the fentanyl negative subpopulation were assigned to RI. Randomization was stratified by the same-day UDS fentanyl result.

### Treatment Retention

In the sub-study, treatment retention at injection 2 was defined as the proportion of participants receiving injection 2 at the week 2 Visit among those in the Evaluable Population for treatment retention/discontinuation. If the participant received injection 2 at the week 2 visit, or missed injection 2 at week 2 but received injection 3 at the week 6/day 36 visit, the participant was retained; otherwise the participant was not retained. The primary endpoint retention rate difference at injection 2 was estimated by Bayesian approach. The posterior probability was evaluated for non-inferiority (NI) of RI to SI using a 10% NI margin against 96% critical value for 1-sided Type I error <10%. Table 4 below presents the results.

TABLE 4
Treatment Retention at Injection 2

| | Fentanyl POS | | Fentanyl NEG | | | |
|---|---|---|---|---|---|---|
| | Subpopulation | | Subpopulation | | Overall | |
| | SI | RI | SI | RI | SI | RI |
| Category | (N = 34) | (N = 60) | (N = 13) | (N = 25) | (N = 47) | (N = 85) |
| Proportion of participants who | 52.9% (18/34) | 65.0% (39/60) | 76.9% (10/13) | 72.0% (18/25) | 59.6% (28/47) | 67.1% (57/85) |
| received Injection 2 (n/N) | | | | | | |
| Difference on retention rate at | | 11.6% | | −2.8% | | 7.5% |
| Injection 2-(RI-SI) (95% HPD) | | (−8.2, 31.7) | | (−30.1, 24.8) | | (−8.7, 24.5) |
| Probability of RI non-inferior to [symbol] I, | | 98.2% | | 68.5% | | 98.2% |
| (RI-SI) > −10% | | | | | | |
| Probability of RI superior to SI | | 87.0% | | 40.8% | | 80.9% |
| (RI-SA) > 0 | | | | | | |

HPD = highest posterior density region;
RI = rapid induction;
SD = standard deviation;
SI = standard of care. [symbol]
indicates data missing or illegible when filed

Table 4 shows that the proportion (percentage) of participants who received injection 2 was 52.9% in the SI arm and 65.0% in the RI arm of the fentanyl positive subpopulation, while the proportion was 76.9% in the SI arm and 72.0% in the RI arm in fentanyl negative population. The proportion was 59.6% in the SI arm and 67.1% in RI overall. Table 4 also shows that probability of RI being non-inferior to SI (RI−SI>−10%) was 98.2% in fentanyl positive population, 68.5% in fentanyl negative population, and 98.2% overall. Finally, Table 4 shows that the probability of RI being superior to SI (RI−SI>0) was 87.0% in the fentanyl positive population, 40.8% in the fentanyl negative population, and 80.9% overall.

As a result, it can be said that the retention rate is lower in the fentanyl positive subpopulation compared to the fentanyl negative population. Also, the observed treatment retention is numerically higher, compared to SI, in the RI arm in both the overall population and among participants who were fentanyl positive on the day of induction. Further, the results indicate that RI is non-inferior to SI since the probability of RI non-inferiority to SI is ≥98%.

### Precipitated Opioid Withdrawal (POW)

The sub-study evaluated opioid withdrawal symptoms and precipitated opioid withdrawal (POW) symptoms of participants up to injection 2. The results are shown in Table 5.

TABLE 5
Precipitated Opioid Withdrawal up to Injection 2

| | n (%) | | | | | | |
|---|---|---|---|---|---|---|---|
| | Fentanyl Positive | | Fentanyl Negative | | Overall | | |
| | (N = 96) | | (N = 41) | | (N = 137) | | |
| | SI | RI | SI | RI | SI | RI | Total |
| Category (Participants with) | (N = 35) | (N = 61) | (N = 13) | (N = 28) | (N = 48) | (N = 89) | (N = 137) |
| TEAE reported as opioid | 9 (25.7) | 15 (24.6) | 1 (7.7) | 2 (7.1) | 10 (20.8) | 17 (19.1) | 27 (19.7) |
| withdrawal symptom | | | | | | | |
| TEAE reported as precipitated | 8 (22.9) | 13 (21.3) | 0 (0.0) | 2 (7.1) | 8 (16.7) | 15 (16.9) | 23 (16.8) |
| opioid withdrawal symptom | | | | | | | |

As seen in Table 5, the number (percentage) of participants who have TEAE reported as opioid withdrawal symptoms was 9 out of 35 participants (25.7%) in the SI arm, and 15 out of 61 participants (24.6%) in the RI arm of the fentanyl positive subpopulation, and 1 out of 13 participants (7.7%) in the SI arm compared to 2 out of 28 participants (7.1%) in the RI arm of the fentanyl negative subpopulation. Overall, 10 out of 48 participants (20.8%) for the SI arm, 17 out of 89 participants (19.1%) for the RI arm, and 27 out of 137 (19.7%) in total had opioid withdrawal symptoms.

Table 5 also shows that the number (percentage) of participants who have TEAE reported as POW symptoms was 8 out of 35 participants (22.9%) in the SI arm and 13 out of 61 participants (21.3%) in the RI arm of the fentanyl positive subpopulation, 0 out of 13 participants (0%) in the SI arm and 2 out of 28 participants (7.1%) in the RI arm of the fentanyl negative subpopulation, and 8 out of 48 participants (16.7%) for the SI arm overall, 15 out of 89 participants (16.9%) for the RI arm overall, and 23 out of 137 (16.8%) in total.

As can be seen from Table 5, more participants who reported POW were fentanyl positive (21/23); thus, POW appears to be a fentanyl-related issue. Most participants reported POW symptoms after TM buprenorphine in RI: n=10 and SI: n=7. All participants who used concomitant medications were in the fentanyl positive subpopulation. The most common medications were clonidine, ondansetron, loperamide, and trazodone. A higher percentage of SI participants (100%) used concomitant medications to treat withdrawal symptoms compared to RI (33.3%) on the first day of induction.

Finally, retention of participants after POW was higher for RI than SI: RI 80.0% vs. SI 37.5% at injection 1; RI 53.3% vs. SI 37.5% at injection 2; and RI 53.3% vs. SI 37.5% at injection 3.

It can be concluded that the incidence of POW is similar between RI and SI, which means that RI does not increase POW. Thus, it can be said that precipitated opioid withdrawal was not more likely for RI. Also, when POW occurred in RI, events were not more serious, severe, or long-lasting, and did not result in an increase in discontinuation compared to SI.

### Clinical Opiate Withdrawal Score (COWS)

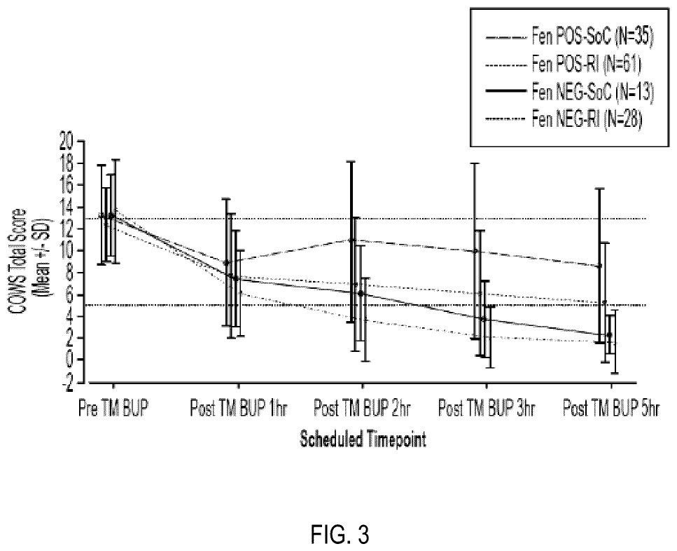

Clinical Opiate Withdrawal Scores (COWS) were evaluated on induction day pre-dose and at 1, 2, 3, and 5 hours post-TM buprenorphine. The results are shown in FIG. 3. As FIG. 3 shows, the response of opioid withdrawal symptoms was comparable for SI and RI overall. COWS scores tended to be lower in the RI arm compared to the SI arm during induction. FIG. 3 also shows that fentanyl positive participants tended to have higher COWS scores during induction.

### Treatment-Emergent Adverse Events (TEAEs)

Treatment-emergent adverse events (TEAEs) were evaluated up to injection 2 in the sub-study. The results are shown in Table 6.

TABLE 6
TEAE Profile Up to Injection 2

| | n (%) | | | | | | |
|---|---|---|---|---|---|---|---|
| | Fentanyl Positive | | Fentanyl Negative | | Overall | | |
| | (N = 96) | | (N = 41) | | (N = 137) | | |
| | SI | RI | SI | RI | SI | RI | Total |
| Category (Participants with) | (N = 35) | (N = 61) | (N = 13) | (N = 28) | (N = 48) | (N = 89) | (N = 137) |
| Any TEAE up to Injection 2 | 14 (40.0) | 20 (32.8) | 4 (30.8) | 6 (21.4) | 18 (37.5) | 26 (29.2) | 44 (32.1) |
| TM BUP-related TEAEs | 7 (20.0) | 8 (13.1) | 0 (0.0) | 2 (7.1) | 7 (14.6) | 10 (11.2) | 17 (12.4) |
| RBP-6000 related TEAEs | 6 (17.1) | 10 (16.4) | 3 (23.1) | 2 (7.1) | 9 (18.8) | 12 (13.5) | 21 (15.3) |
| Serious TEAEs | 0 (0.0) | 1 (1.6) | 0 (0.0) | 0 (0.0) | 0 (0.0) | 1 (1.1) | 1 (0.7) |
| TEAEs leading to | 0 (0.0) | 2 (3.3) | 0 (0.0) | 2 (7.1) | 0 (0.0) | 4 (4.5) | 4 (2.9) |
| discontinuation | | | | | | | |

As seen in Table 6, 35 participants of the SI arm and 61 participants of the RI arm among fentanyl positive subpopulation, and 13 participants of the SI arm and 28 participants of the RI arm among fentanyl negative population, are categorized. Table 6 shows that any TEAE up to injection 2 was 14 out of 35 participants in the SI arm compared to 20 out of 61 participants in the RI arm for the fentanyl positive population, and 4 out of 13 participants in the SI arm compared to 6 out of 28 participants in the RI arm in the fentanyl negative population. There were 18 out of 48 participants in the SI arm overall, 26 of 89 participants in the RI arm overall, and 44 out of 137 in total, with TEAEs. The number of participants who experienced received TM buprenorphine-related TEAEs was 7 out of 35 participants in the SI arm, 8 out of 61 participants in the RI arm in the fentanyl positive population, and 0 out of 13 participants in the SI arm compared to 2 out of 28 participants in the RI arm in the fentanyl negative population, 7 out of 48 participants in the SI arm overall, 10 of 89 in the RI arm overall, and 17 out of 137 in total.

The number of participants who experienced BUP-XR related TEAEs was 6 out of 35 participants in the SI arm compared to 10 out of 61 participants in the RI arm in the fentanyl positive population and 3 out of 13 participants in the SI arm compared to 2 out of 28 in the RI arm in the fentanyl negative population, with 9 out of 48 participants in SI overall, 12 of 89 in RI overall, and 21 out of 137 in total. The number of participants who experienced serious TEAEs was 0 out of 35 participants in the SI arm compared to 1 out of 61 participants in the RI arm in the fentanyl positive population and 0 out of 13 participants in the SI arm compared to 0 out of 28 participants in the RI arm in the fentanyl negative population, with 0 out of 48 participants in SI overall, 1 of 89 in RI overall, and 1 out of 137 in total. The number of participants experiencing TEAEs leading to discontinuation was 0 out of 35 participants in the SI arm and 2 out of 61 participants in RI arm in the fentanyl positive population, and 0 out of 13 participants in the SI arm and 2 out of 28 participants in the RI arm in the fentanyl negative population, with 0 out of 48 participants in SI overall, 4 of 89 in RI overall, and 4 out of 137 in total.

In SI, up to injection 3, it can be said that administration of BUP-XR injection 2 as soon as one week (rather than four weeks) after injection 1 (day 1 for both RI and SI) was well tolerated. There were some adverse events from this study of RI and SI. For example, there was a small decline in the number of participants reporting TEAEs before 300 mg of BUP-XR compared to after 300 mg of BUP-XR. However, there was no individual adverse event observed in ≥5% of participants between injections 2 and 3.

This sub-study demonstrates that, compared to standard induction, rapid induction had comparable outcomes for treatment retention and precipitated opioid withdrawal in this high-risk population. Administration of injection 2 was safe and well tolerated. The shorter time necessary for RI may increase feasibility of BUP-XR treatment.

### Final Analysis

Once the study was complete, final analysis of the data shows that RI treatment is surprisingly superior to SI treatment. Table 7A below shows disposition of all randomized participants, and Table 7B shows disposition of the safety full analysis set (SFAS) through injection 3. Of the 785 randomized participants, 729 (92.9%) were included in the SFAS.

TABLE 7A
Disposition of Randomized Participants

| | Overall (n = 785) | |
|---|---|---|
| | SI (n = 267) | RI (n = 518) |
| Induction (TM BUP) (%) | 264 (98.9%) | 513 (99.0%) |
| Week 1; Injection 1 (%) | 159 (59.6%) | 444 (85.7%) |
| Week 2; Injection 2 (%) | 145 (54.3%) | 339 (65.4%) |
| Week 6; Injection 3 (%) | 137 (51.3%) | 298 (57.5%) |

TABLE 7B
Disposition of SFAS Participants

| | Fentanyl Positive | | Fentanyl Negative | | Overall | |
|---|---|---|---|---|---|---|
| | N = 563 | | N = 166 | | n = 729* | |
| | SoC | RI | SoC | RI | SoC | RI |
| Category (n) | (N = 196) | (N = 367) | (N = 59) | (N = 107) | (N = 255) | (N = 474) |
| Induction (TM BUP) (%) | 100.0% | 100.0% | 100.0% | 100.0% | 100.0% | 100.0% |
| Injection 1 (%) | 52.0% | 84.5% | 83.1% | 92.5% | 151 (59.2%) | 409 (86.3%) |
| Injection 2 (%) | 47.4% | 62.4% | 76.3% | 79.4% | 138 (54.1%) | 314 (66.2%) |
| Injection 3 (%) | 44.4% | 56.4% | 71.2% | 68.1% | 129 (50.6%) | 278 (58.6%) |

*Includes the 6 non-eligible participants who were discontinued.

### Treatment Retention

With respect to participant retention through injection 2 (participants receiving injection 2 who received at least 1 dose of TM buprenorphine for induction and did not demonstrate idiosyncratic response to the first dose of TM buprenorphine), the RI arm showed superiority to the SI arm. The retention results are summarized in Table 8.

TABLE 8
Treatment Retention at Injection 2

| | Fentanyl Positive | | Fentanyl Negative | | Overall | |
|---|---|---|---|---|---|---|
| | N = 560 | | N = 163 | | N = 723* | |
| | SoC | RI | SoC | RI | SoC | RI |
| Category | (N = 194) | (N = 366) | (N = 59) | (N = 104) | (N = 253) | (N = 470) |
| Proportion of participants who | 47.9% (93/194) | 62.8% (230/366) | 76.3% (45/59) | 78.8% (82/104) | 54.5% (138/253) | 66.4% (312/470) |
| received injection 2 (n/m) | | | | | | |
| Difference on retention rate at | | 14.84% | | 2.96% | | 11.82% |
| injection 2-(RI-SoC) (95% HPD) | | (6.51, 23.72)% | | (−10.50, 16.10)% | | (4.34, 19.03)% |
| Posterior probability of RI non- | | 100.00% | | 87.45% | | 100.00% |
| inferior to SoC, ie, (RI-SoC) > −10% | | | | | | |
| Posterior probability of RI superior to | | 99.98% | | 66.32% | | 99.90% |
| SoC, ie, (RI-SoC) > 0 | | | | | | |

HPD = highest posterior density region;
SD = standard deviation;
SoC = standard of care;
RI = rapid induction
*Evaluable for treatment retention population

As Table 8 shows, RI treatment was superior to SI treatment with respect to participant retention through injection 2. The proportion of participants who received injection 2 was higher in the RI arm (66.4%) compared with the SI arm (54.5%), and the estimated treatment retention rates at injection 2 were 66.3% and 54.5%, respectively. The difference (95% highest posterior density (“HPD”)) in the retention rate at injection 2 was 11.82% (4.34%, 19.03%) in favor of RI treatment.

The results for the fentanyl positive subpopulation were supportive of the overall results (RI=62.8% and SI=47.9%; estimated treatment retention rates at injection 2=62.7% and 47.9%, respectively; difference [95% HPD]=14.84% [6.51%, 23.72%]), while in the fentanyl negative subpopulation, the treatment retention rates for the two treatment arms were numerically comparable (SI=75.3% and RI=78.3%). When the primary endpoint was analysed as “randomized,” as well as in additional sensitivity analyses, the results were very similar to those in the primary analysis, demonstrating the robust nature of these findings.

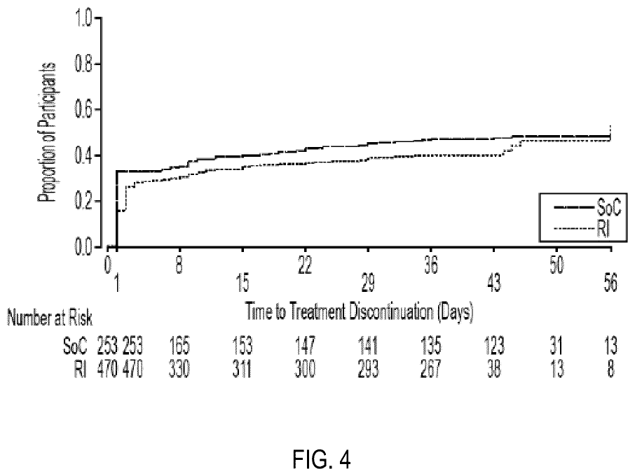

Additionally, as shown in FIG. 4, the time to treatment discontinuation among those in the evaluable population for treatment retention/discontinuation favored the RI treatment arm compared with the SI treatment arm, with hazard ratio=0.82 for RI/SI, 95% CI (0.65, 1.03), accounting for the fentanyl UDS subpopulation. Even though the SI arm included an extra week of TM buprenorphine induction compared with the RI arm, the retention rate at 5 weeks post-injection 1 was higher in the RI arm compared with the SI arm (60.2% and 53.4%, respectively).

### Precipitated Opioid Withdrawal (POW) and Clinical Opioid Withdrawal Score (COWS)

The evaluation of POW was an exploratory endpoint. Per protocol, investigators were instructed to evaluate whether POW occurred and, if so, to document any associated adverse events. An additional analysis was conducted in which POW was defined as an increase of at least 6 points in the COWS total score, measured within five hours following TM buprenorphine administration. Thus, the evaluation focused on precipitated withdrawal based on both the investigator assessment and the COWS score increase of at least 6 points. Table 9 below presents a comparison of POW events between the RI arm and the SI arm.

TABLE 9
Comparison of POW Events

| | n (%) | | | | | |
|---|---|---|---|---|---|---|
| | Fentanyl Positive | | Fentanyl negative | | Overall | |
| | N = 563 | | N = 166 | | N = 729 | |
| | SoC | RI | SoC | RI | SoC | RI |
| POW Category (Participants with) | (N = 196) | (N = 367) | (N = 59) | (N = 107) | (N = 255) | (N = 474) |
| Investigator Assessed | 44 (22.4) | 107 (29.2) | 2 (3.4) | 7 (6.5) | 46 (18.0) | 114 (24.1) |
| RI-SoC (95% CI) | | | | | 6.01% (−0.30%, 11.92%) | |
| COWS increase ≥6 | 27 (13.8) | 51 (13.9) | 2 (3.4) | 2 (1.9) | 29 (11.4) | 53 (11.2) |
| Investigator Assessed AND COWS | 16 (8.2) | 34 (9.3) | 0 | 1 (0.9) | 16 ( [symbol] .3) | 35 (7.4) |
| increase ≥6 | | | | | | |

[symbol]

indicates data missing or illegible when filed

As shown in Table 9, the percentage of participants who experienced POW during induction was similar between treatment arms, whether analyzed based on investigator assessment of POW or on a COWS score increase of at least 6 points. Importantly, retention of participants with investigator-assessed POW was higher for RI than for SI: at injection 2, the difference in retention was 13.72% (95% CI −2.3, 29.8).

Most participants who experienced POW were fentanyl positive at baseline. Most treatment-emergent adverse events (TEAEs) associated with POW were of mild or moderate intensity. Among participants who experienced POW, retention through injection 2 and injection 3 was higher for the RI arm than for the SI arm.

While there is currently no universally accepted definition of precipitated withdrawal, recent publications suggest that an increase in COWS of at least 6 is a reasonable and objective cut point for precipitated withdrawal. Similar percentages of participants in the SI (11.4%) and RI (11.2%) arms had a COWS total score increase from pre-TM buprenorphine of at least 6 on the most recent induction day at any time within the five hours on site.

With respect to TEAEs associated with precipitated withdrawal, similar percentages of participants in the SI and RI arms reported TEAEs associated with precipitated withdrawal post TM buprenorphine (16.1% and 12.7% respectively) and post BUP-XR in the RI arm (13.9%), while a lower percentage of participants in the SI arm experienced these events (4.0%). These events were more common in the fentanyl positive subpopulation compared with the fentanyl negative subpopulation.

Most participants in the RI arm, even those who experienced events of precipitated withdrawal after TM buprenorphine, received BUP-XR within 1 to 2 hours after the first TM buprenorphine administration, so withdrawal symptoms did not result in delayed BUP-XR injection.

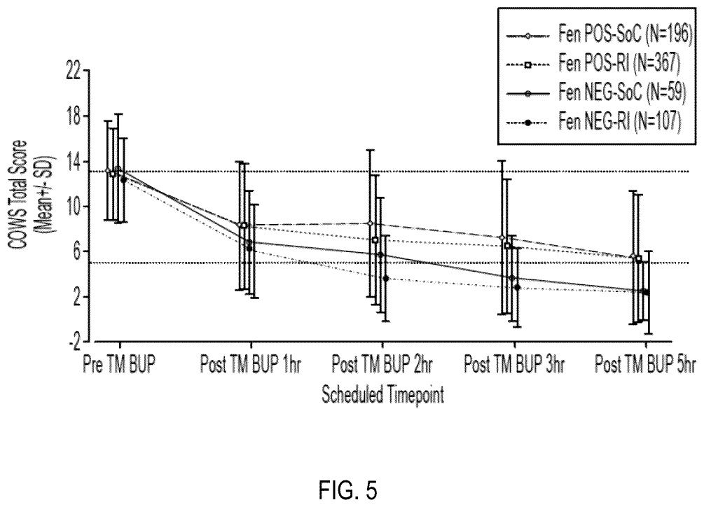

On induction day, the response of opioid withdrawal symptoms, as measured by COWS total scores, was comparable for both the SI and RI arms overall and within the fentanyl subpopulation, although symptoms were more pronounced in participants in the fentanyl positive subpopulation. This is seen in FIG. 5. Participants in the RI arm received TM buprenorphine followed by BUP-XR at 1 hour, while those in the SI arm received only TM buprenorphine. In most individuals, the COWS total score peaked at 2 to 3 hours post-TM buprenorphine, which corresponds to 1 to 2 hours post-injection in the RI group. These findings suggest that observation for 3 hours post-TM buprenorphine (i.e., 2 hours post-BUP-XR) could be recommended for RI.

In a comparison of treatment arms, the percentage of participants experiencing POW was similar for both the RI and SI arms, with no significant differences in investigator-assessed or changes in COWS events. Most POW events were rated as mild or moderate in severity, with less than 2% classified as severe, and no participants recorded COWS scores in the severe range (>36). However, a marginally higher percentage of participants in the SI arm exhibited moderately severe scores (25-36) compared to those in the RI arm. Regarding the use of concomitant medications to manage withdrawal symptoms, 80.5% of the SI arm and 72% of the RI arm utilized such treatments, with clonidine, ondansetron, and trazodone being the most commonly prescribed.

### Treatment-Emergent Adverse Events (TEAEs)

Overall TEAE results of the study are shown in Table 10.

TABLE 10
Overall Treatment-Emergent Adverse Events to Injection 2

| | n (%) | | | | | | |
|---|---|---|---|---|---|---|---|
| | Fentanyl Positive | | Fentanyl Negative | | Overall | | |
| | N = 563 | | N = 166 | | N = 729 | | |
| | SoC | RI | SoC | RI | SoC | RI | Total |
| Category (Participants with) | (N = 196) | (N = 367) | (N = 59) | (N = 107) | (N = 255) | (N = 474) | (N = 729) |
| Any TEAE up to Injection 2 | 72 (36.7) | 164 (44.7) | 21 (35.6) | 38 (35.5) | 93 (36.5) | 202 (42.6) | 295 (40.5) |
| Severe TEAEs | 6 (3.1) | 15 (4.1) | 0 | 2 (1.9) | 6 (2.4) | 17 (3.6) | 23 (3.2) |
| Serious TEAES | 1 (0.5) | 5 (1.4) | 0 | 0 | 1 (0.4) | 5 (1.1) | 6 (0.8) |
| TEAEs potentially related to | 1 (0.5) | 1 (0.3) | 0 | 0 | 1 (0.4) | 1 (0.2) | 2 (0.3) |
| Hepatic disorders | | | | | | | |
| TEAE reported as Opioid | 55 (28.1) | 137 (37.3) | 9 (15.3) | 12 (11.2) | 64 (25.1) | 149 (31.4) | 213 (29.2) |
| withdrawal symptom | | | | | | | |
| TM BUP-related TEAEs | 45 (23.0) | 68 (18.5) | 9 (15.3) | 7 (6.5) | 54 (21.2) | 75 (15.8) | 129 (17.7) |
| TEAEs leading to | 1 (0.5) | 2 (0.5) | 0 | 0 | 1 (0.4) | 2 (0.4) | 3 (0.4) |
| discontinuation from TM BUP | | | | | | | |

Table 10 shows the overall and TM buprenorphine TEAE profile up to injection 2 was comparable for the RI and SI arms. The percentage of participants in the SFAS with TEAEs up to injection 2 was 36.5% in the SI arm compared with 42.6% in the RI arm (% difference RI−SoC [95% CI]=6.1 [−1.3, 13.4]; among participants in the SFAS who actually received BUP-XR, these values were 40.4% and 40.8% (0.4 [−8.8, 9.4]). The percentages of participants in the SFAS with TEAEs up to injection 2 were not different between treatment arms in the fentanyl UDS subpopulations: 36.7% in the SI arm and 44.7% the RI arm in the fentanyl positive subpopulation (% difference RI-SoC [95% CI]=8.0 [−0.6, 16.2]) and 35.6% and 35.5%, respectively, in the fentanyl negative subpopulation (−0.1 [−15.6, 14.6]).

Most TEAEs up to injection 2 were of mild or moderate intensity. Severe TEAEs were reported in low and similar percentages of participants in the SI and RI arms (2.4% and 3.6%, respectively; % difference RI-SoC [95% CI]=1.2 [−1.7, 3.7]).

Serious TEAEs up to injection 2 were reported for 1 participant (0.4%) in the SI arm and 5 participants (1.1%) in the RI arm, all in participants in the fentanyl positive subpopulation (% difference RI-SoC [95% CI]=0.7 [−1.2, 2.1]). None of these was fatal or met the liver function test criterion of ALT or AST values>3×ULN (upper limit of normal) and bilirubin>2×ULN. Two participants, one in each treatment arm, had TEAEs potentially related to hepatic disorders (per customized Medical Dictionary for Regulatory Activities query definition).

Three participants discontinued TM buprenorphine as a result of TEAEs up to injection 2 (SI=1 participant [0.4%] and RI=2 participants [0.4%]; % difference RI-SoC [95% CI]=0.0 [−1.8, 1.2]). Among participants in the SFAS who actually received BUP-XR, 5 participants (all RI, 1.2% among those in the RI who received BUP-XR) discontinued BUP-XR as the result of a TEAE up to injection 2 (0.3 [−1.8, 1.8]).

There was a notable decline in TEAEs between injections 2 and 3 compared to the rates observed up to injection 2. Specifically, the percentage of participants experiencing TEAEs between injections 2 and 3 was lower for both the SI arm (21.0% vs. 36.5%) and the RI arm (27.4% vs. 42.6%). The TEAE profile remained consistent across both SI and RI arms. Additionally, two participants experienced serious adverse events (SAEs), one of which was fatal, and two participants had TEAEs that led to discontinuation from BUP-XR; however, none of these events were related to the medication.

The results of this study surprisingly demonstrate that RI treatment was superior to SI treatment regarding participant retention through injection 2. The RI regimen was found to be safe and well tolerated, with POW events not being more likely to occur or be more severe with the RI regimen than with the SI regimen. Notably, most POW events (151 of 160) occurred in participants who were fentanyl positive at baseline. Among those who experienced precipitated withdrawal, retention rates through injection 2 and injection 3 were higher for the RI group compared to the SoC group. Additionally, the administration of BUP-XR injection 2 as soon as 1 week (rather than 4 weeks) after injection 1 was well tolerated under the conditions of this study, and pharmacokinetic data support similar plasma exposure to buprenorphine when compared to standard administration at four weeks.

1. A method of treating opioid use disorder in a human in need thereof, the method comprising:
(a) administering a composition comprising transmucosal buprenorphine hydrochloride to the human on day 1, and
(b) administering a second composition comprising about 300 mg of BUP-XR by subcutaneous injection to the human at least 1 hour later.
